(19) United States
(12) Patent Application Publication
(10) Pub. No.: US 2010/0035933 A1
(43) Pub. Date: Feb. 11, 2010

(54) Kits, Recloseable Containers, Blanks And Methods Of Treatment

(76) Inventors: Will Howard Mitchell, Loveland, OH (US); Linda Carol Jones, Aurora, IN (US); John Stephen Eadicicco, West Chester, OH (US); Beth Waggoner Russell, Goshen, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/564,366

(22) Filed: Sep. 22, 2009

Related U.S. Application Data
(63) Continuation-in-part of application No. 10/922,260, filed on Aug. 19, 2004.
(60) Provisional application No. 60/496,423, filed on Aug. 20, 2003.

(51) Int. Cl. A61K 31/4439; B65D 71/00; B65D 5/00

(52) U.S. Cl. 514/338; 206/570; 229/126

(57) ABSTRACT

Kits include a recloseable container and an inner container housed within the recloseable container. The recloseable container can include a front wall and a front flap operable to transition between an open position and a closed position, wherein the front flap releasably engages the front wall when in the closed position. Methods of treatment which utilize the kits are also disclosed. Blanks for the recloseable container are also disclosed.

### CROSS REFERENCE TO RELATED APPLICATION

This application is a continuation-in-part of U.S. patent application Ser. No. 10/922,260, filed Aug. 19, 2004 which claims the benefit of U.S. Provisional Application No. 60/496,423, filed Aug. 20, 2003.

### FIELD OF THE INVENTION

The invention relates to a kits, recloseable containers, blanks, and methods useful for treatment of upper gastrointestinal conditions.

### BACKGROUND OF THE INVENTION

User compliance with treatment regimens remains a significant healthcare issue. Compliance is further significant when medications are approved for over-the-counter (OTC) sale, as this provides the user with increased access to medications. Even further, compliance with upper gastrointestinal conditions, for example frequent heartburn, presents some particularly unique issues. Because of the pain and unpleasantness of the symptoms, users may require little encouragement to begin a treatment regimen. In some users, however, symptoms are persistent or, in contrast, transient. If may be desirable to inform these users regarding the optimal course of treatment for the condition.

Frequent heartburn results from gastric acid being released into the lower esophagus. The user usually experiences a burning chest pain, which begins behind the breastbone and moves upward to the neck and throat. Gastrointestinal Disease, Pathophysiology, Diagnosis and Management, 5^{th} Ed., Vol. 1, B. Scharschmidt and M. Feldman, Eds., pp. 378-79 (1993). Proton pump inhibitors (PPIs) are a class of pharmaceutical compounds that effectively prevent or inhibit gastric acid secretion by inhibiting the H⁺/K⁺-ATP enzyme system. Drug Facts and Comparisons, Cada et al. Eds., pp. 1137-38 (May 2003). Omeprazole is one example of a PPI that has proven effective in the prevention and treatment of upper gastrointestinal conditions, including frequent heartburn, GERD, gastritis, gastric ulcer and duodenal ulcer. See e.g., U.S. Pat. Nos. 4,508,905; 4,738,974; and 5,900,424.

A composition containing omeprazole has been approved for OTC sale for the treatment of frequent heartburn. This raises the aforementioned complex user compliance issue.

A number of references describe treatment regimens for certain medications and seek to increase user compliance therewith. For example, many are directed to the administration of bisphosphonates, which belong to the class of compounds known as bone resorption inhibitors. See e.g., U.S. Pat. Nos. 4,761,406; 5,616,560; 5,994,329. Bisphosphonates are prescribed for the treatment of chronic conditions, such as osteoporosis, in which there is a continual loss of bone due to resorption. Strict adherence to these dosing regimens is necessary for effective treatment, and user compliance is an important concern. See e.g., U.S. Pat. 5,366,965 and Drug Facts and Comparisons, Cada et al., Eds., p. 584 (May 2003, updated monthly).

The above dosing regimens can be either continuous or cyclic. Continuous dosing regimens involve administration of relatively low doses of bisphosphonates at regular intervals. One problem with continuous dosing of bisphosphonates, however, is that the body adjusts to the attempts to regulate bone resorption, and may counteract any gain in bone mass. Cyclic dosing regimens were developed to address this problem, and require administration of higher doses of bisphosphonates during a given time interval, followed by a rest period during which no drug is administered. Without intending to be limited by theory, the physiological basis for the rest period is to uncouple bone formation and resorption by selectively inhibiting the resorption phase of bone remodeling without appreciably affecting the formation phase. Disorders of Bone and Mineral Metabolism, Fredric L. Coe and Murry J. Favus, Eds., 866-67 (1992); U.S. Pat. No. 4,761,406. As such, the success of these therapies is uniquely dependent upon the prescribed rest period.

Several references also teach kits that are designed to increase user compliance with these and other treatment regimens. See e.g., U.S. Pat. Nos. 4,534,468; 4,889,237; 5,833,072; and U.S. Patent Publication 2001/0044427 A1.

However, there is a continuing need to address the more complex user compliance issues that arise from over-the-counter sale of medication for the treatment of gastrointestinal conditions. For example, the aforementioned dosing regimens and kits fail to address the problem of encouraging responsible use of over-the-counter medications to treat acute symptoms of gastrointestinal disorders.

### SUMMARY OF THE INVENTION

The present disclosure further relates to kits, recloseable containers, blanks and methods useful for facilitating compliance with a treatment regimen.

In one or more embodiments, a convenient kit optionally designed to facilitate user compliance with a flexible dosing regimen is shown and described herein. The treatment regimen provides for dosing periods during which a sufficient number of doses of a gastric acid secretion inhibitor are administered to provide relief from symptoms. The one or more embodiments herein provides for dosing according to a discontinuous schedule. In a discontinuous schedule, each dosing period is followed by an evaluation period, during which the user can self-evaluate the occurrence and severity of symptoms. If the user determines that it is necessary to begin a new dosing period, the user may do so at any time following this evaluation period. If, however, the user feels the need to begin a new dosing period before the evaluation period has passed, or to take more than the recommended number of doses, then the user may, for example, choose to seek professional medical advice.

The kits are designed to encourage compliance with the dosing and to the evaluation periods. In one embodiment, one or more containers comprising individually-packaged dosing regimens are contained in a single kit, and more specifically, in a recloseable container. Each inner container contains a sufficient number of doses of a gastric acid secretion inhibitor for one dosing period. Instructions, including optionally motivational text, may be further disposed about the recloseable container and/or the inner container(s). To begin a new dosing period, the user therefore opens the recloseable container and removes an inner container while encountering the instructions, including optionally motivational text. The inclusion of a recloseable container with instructions regarding individually packaged dosing regimens serves as a signal to remind the user that a new treatment regimen may not be undertaken until sufficient time for self-evaluation of symptoms has passed.

In one embodiment, a kit includes a recloseable container and an inner container housed within the recloseable container. The recloseable container may include a front wall and a front flap operable to transition between an open position and a closed position, wherein the front flap may releasably engage the front wall when in the closed position to maintain the recloseable container in the closed position. The inner container includes a plurality of unit doses of a composition comprising a therapeutically effective amount of a gastric acid secretion inhibitor and instructions for compliance with a course of treatment. The instructions include that at least 2 unit doses of the composition should be administered over a dosing period of from about 2 days to about 28 days and that the course of treatment may be repeated after an evaluation period of from about 2 days to about 135 days.

In another embodiment, a kit includes a recloseable container and an inner container. The recloseable container may include a front wall and a front flap operable to transition between an open position and a closed position, wherein the front flap may releasably engage the front wall when in the closed position and wherein an interior surface of the recloseable container comprises instructions for compliance with a course of treatment. The instructions include that at least 2 unit doses of a composition comprising a therapeutically effective amount of a gastric acid secretion inhibitor should be administered over a dosing period of from about 2 days to about 28 days and that the course of treatment may be repeated after an evaluation period of from about 2 days to about 135 days. The inner container includes a plurality of unit doses.

In yet another embodiment, a method for administering a plurality of dosing periods with evaluation periods there between includes providing a recloseable container housing an inner container, wherein the inner container comprises a plurality of unit doses of a composition comprising a therapeutically effective amount of a gastric acid secretion inhibitor, and providing on an interior surface of the recloseable container instructions for compliance with a course of treatment. The instructions include that at least 2 unit doses of the composition should be administered over a dosing period of from about 2 days to about 28 days and that the course of treatment may be repeated after an evaluation period of from about 2 days to about 135 days.

In still yet another embodiment, a blank for a recloseable container may include a front wall connected to a first side of a back wall by a bottom wall, a front flap connected to a second side of the back wall opposite the first side by a top wall, the front flap operable to removably engage a slit in the front wall when in a constructed position, and instructions for compliance with a course of treatment. The instructions include that at least 2 unit doses should be administered over a dosing period of from about 2 days to about 28 days and that the course of treatment may be repeated after an evaluation period of from about 2 days to about 135 days.

These and other embodiments of the invention are described herein.

### DETAILED DESCRIPTION OF THE FIGURES

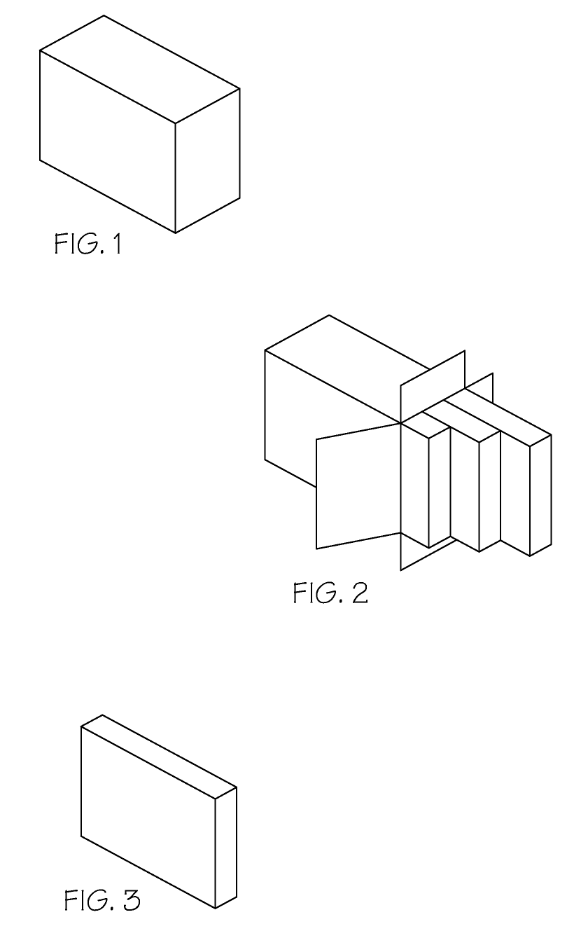

Presented for convenience, FIG. 1 is an illustrative example of an outer container in accordance with the present invention.

Also presented for convenience, FIG. 2 is an illustrative example of the outer container containing three separate inner containers. In an embodiment herein, the inner containers each comprise substantially similar instructions.

Also presented for convenience, FIG. 3 is an illustrative example of one of the separate inner containers also depicted in FIG. 3.

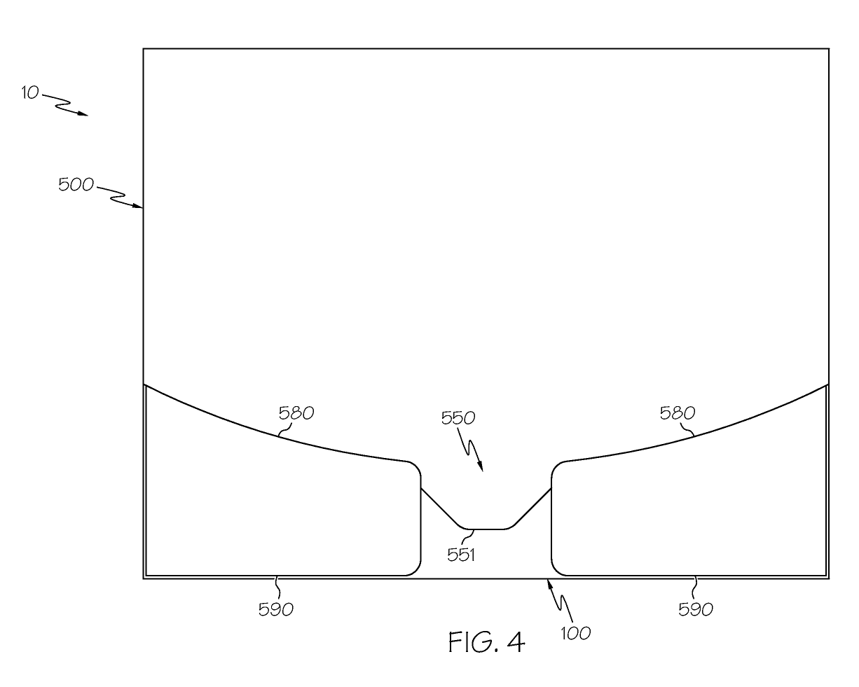

Also presented for convenience, FIG. 4 is an illustrative example of an outer container that comprises a recloseable container in a sealed position containing a plurality of inner containers.

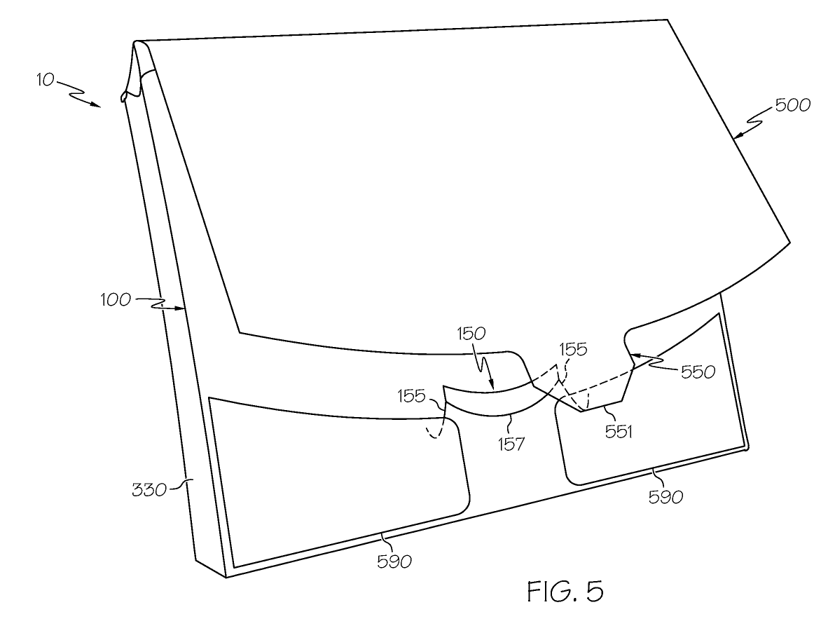

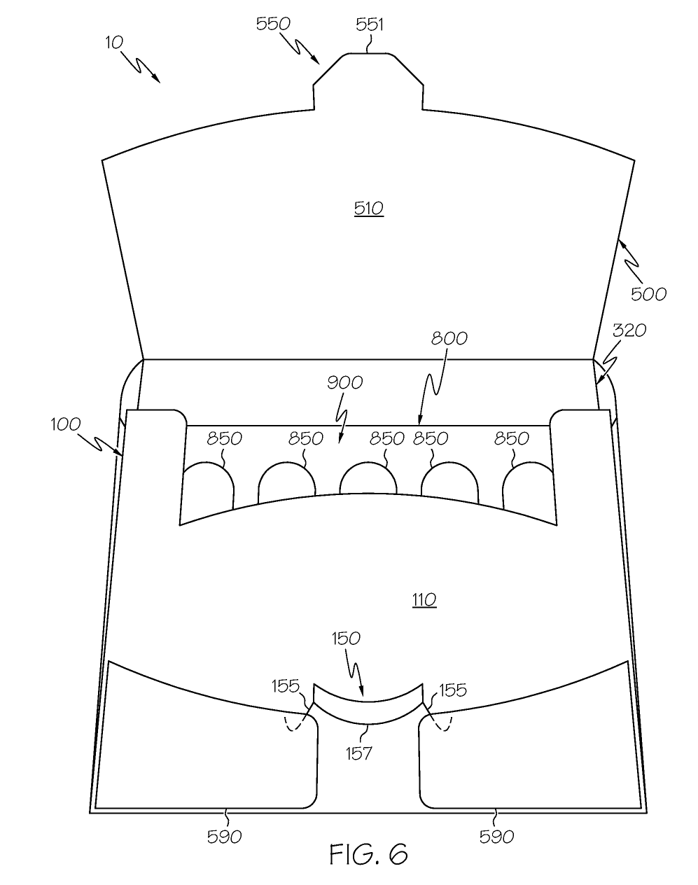

Also presented for convenience, FIG. 5 is an illustrative example of the recloseable container in a partially open position.

Also presented for convenience, FIG. 6 is an illustrative example of the recloseable container in a more fully open position.

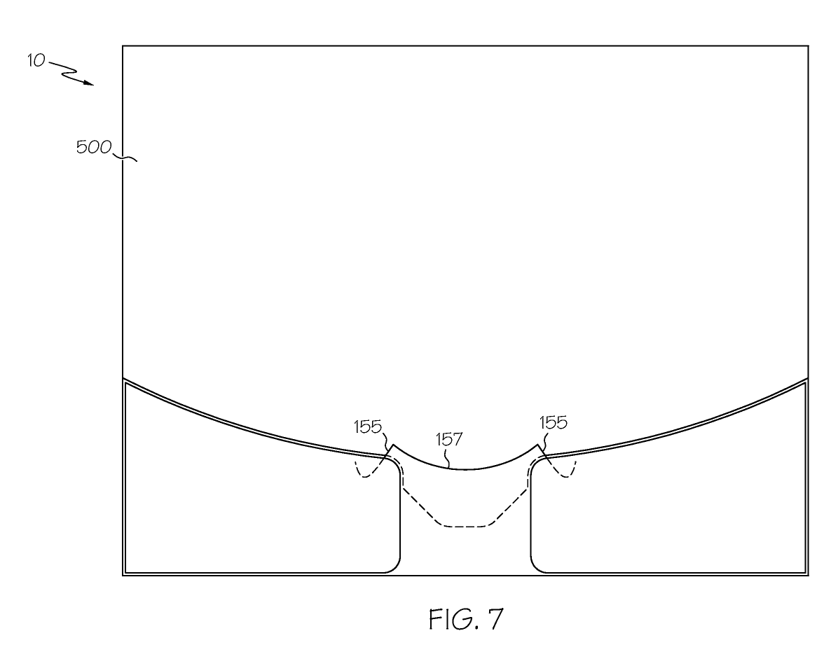

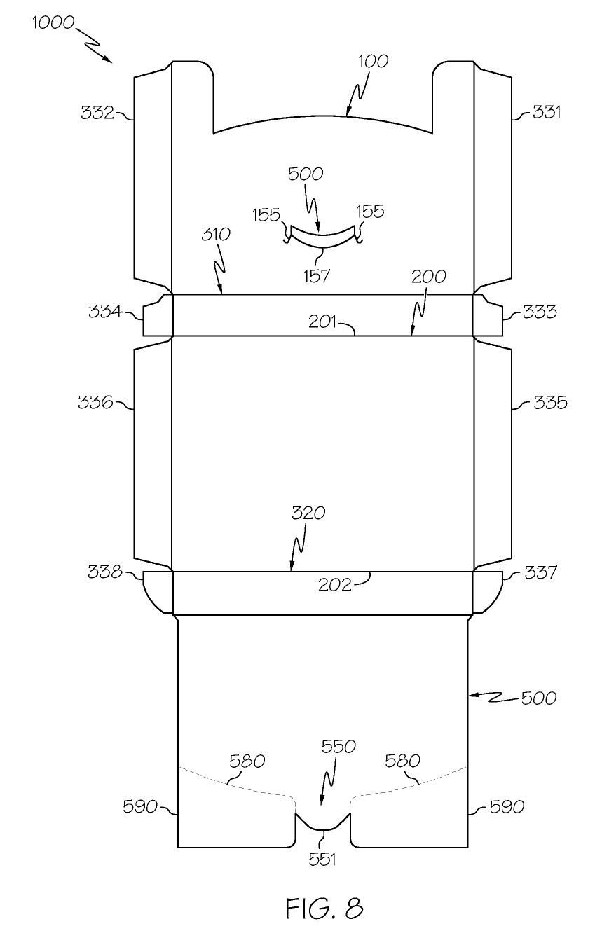

Also presented for convenience, FIG. 7 is an illustrative example of the recloseable container in a closed position.

Also presented for convenience, FIG. 8 is an illustrative example of a blank that may be constructed into a recloseable container.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments or individual features are disclosed. As will be apparent to the ordinarily skilled practitioner, all combinations of such embodiments and features are possible and can result in preferred executions of the present invention.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

The methods and kits herein utilize a composition comprising a gastric acid secretion inhibitor. As used herein, gastric acid secretion inhibitor refers to any compound possessing a cytoprotective and/or gastric acid anti-secretory effect, including, but not limited to, proton pump inhibitors such as omeprazole, esomeprazole, lansoprazole, pantoprazole, and rabeprazole, as well as histamine H₂-antagonists such as ranitidine, cimetidine, nazatidine and famotidine, and mixtures of any of the above. In one embodiment, the methods and kits utilize omeprazole. To illustrate, the omeprazole may be either omeprazole or an omeprazole salt. Examples of salts include omeprazole lithium salts, omeprazole sodium salts, omeprazole potassium salts, omeprazole magnesium salts, omeprazole calcium salts, or mixtures thereof. In another embodiment, the omeprazole salt is selected from omeprazole magnesium salts and omeprazole calcium salts, with omeprazole magnesium salts being among the most preferred. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

Optionally, the composition, or kit, may contain active components other than the gastric acid secretion inhibitor. For example, an antacid may be useful herein. As an example, a composition utilized herein may comprise a gastric acid secretion inhibitor as well as an antacid. As another example, the kits may comprise distinct compositions, wherein a first composition comprises a gastric acid secretion inhibitor and a second composition comprises an antacid. See e.g., U.S. Pat. Nos. 5,385,739; 5,840,737; 6,090,412; 6,183,776; 6,489,346; and 6,551,621; WO 97/25066; WO 00/26185; and EP 0,338,861.

The composition described herein comprises the gastric acid secretion inhibitor, optionally but preferably with a therapeutically acceptable carrier. “Therapeutically acceptable carrier,” as used herein, refers to one or more compatible solid or liquid filler diluents or encapsulating substances which are suitable for administration to a mammal in need thereof, preferably a human. The term “compatible,” as used herein, means that the solid or liquid filler diluents are capable of being combined with the gastric acid secretion inhibitor and with each other, in a manner such that there is no interaction that would substantially reduce the pharmaceutical efficacy of the composition under ordinary use situations. Such therapeutically effective carriers are selected according to criteria well-known to those ordinarily skilled in the art. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

The composition of the present invention is preferably administered in the form of unit doses. As used herein, a “unit dose” contains a therapeutically effective amount of the gastric acid secretion inhibitor, and is suitable for administration to a mammal in need thereof, preferably a human, in a single dose. As an example, each unit dose of the composition may be in the form of either a tablet or capsule, optionally wherein the form comprises some enteric coating (for example, an enteric coating surrounding the tablet or capsule itself or utilizing microencapsulation of the gastric acid secretion inhibitor). As an example, a unit dose of a composition comprising omeprazole may be in the form of a capsule. As another example, a unit dose of a composition comprising an omeprazole magnesium salt may be in the form of a tablet. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

As used herein, the term “therapeutically effective amount,” with reference to a gastric acid secretion inhibitor, means that amount of the inhibitor sufficient to provide a significant improvement of the relevant gastrointestinal condition in a mammal, preferably a human, in need of treatment, yet low enough to avoid adverse effects (such as toxicity, irritation, or allergic response), commensurate with a reasonable benefit/risk ratio when used in the manner of the present invention. The specific “therapeutically effective amount” will vary with such factors as the particular condition being treated, the physical condition of the user, the duration of treatment, the nature of concurrent therapy (if any), the specific dosage form to be used, the carrier employed, the solubility of the dose form, and the particular dosing regimen. Therapeutically effective amounts may comprise from about 5 mg to about 80 mg, from about 5 mg to about 40 mg, from about 5 mg to about 35 mg, or from about 10 mg to about 25 mg of the gastric acid secretion inhibitor in each unit dose. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

The mammals treated herein include, but are not limited to, humans. The most preferred embodiment of which is humans in need of treatment for upper gastrointestinal tract conditions.

As used herein, upper gastrointestinal tract conditions include, but are not limited to, gastroesophageal reflux disease (GERD), erosive esophagitis, gastritis, gastric ulcers, duodenal ulcers, heartburn (including frequent heartburn), indigestion, posterior laryngitis, hypersecretory conditions, such as Zollinger-Ellison syndrome, multiple endocrine adenomas and systemic mastocytosis, and other diseases or disorders in which cytoprotective and/or gastric acid anti-secretory effect is desirable, such as in users with gastrinomas or acute upper gastrointestinal bleeding, or to enhance the efficacy of pancreatin.

### Kits

Several embodiments shown and described herein relate to kits. In one embodiment, the kit comprises a plurality of unit doses of the gastric acid secretion inhibitor and instructions for complying with a treatment method as described herein. Alternatively or additionally, the kit comprises a recloseable container housing one or more inner containers, each inner container containing at least two unit doses of a therapeutically effective amount of the gastric acid secretion inhibitor.

In one embodiment herein, the kit comprises the recloseable container and instructions for complying with a treatment method as described herein. Alternatively or additionally, in another embodiment, each inner container, independently, is a box, sealed pouch, blister pack or other similar container of a suitable size that fits within the recloseable container. In yet another embodiment, each inner container is a box. In yet another embodiment, each inner container is a blister pack. In still yet another embodiment, each inner container is substantially similar (for example, with respect to shape, dimensions, or the like) to each of the other inner container(s) contained within the recloseable container. Alternatively or additionally, each inner container comprises instructions for complying with the treatment method described herein. Thus, in one embodiment, the user of the kit is reminded of such instructions for compliance each time a different inner container is removed from the recloseable container.

In one embodiment, the recloseable container contains a single inner container. In another embodiment, the recloseable container contains a plurality of inner containers such as, for example, two inner containers; in another example, three inner container; in another example, four inner containers; in another example, five inner containers; and so forth. Alternatively or additionally, the recloseable container contains a number of inner containers which corresponds to the anticipated number of dosing periods which the user will experience (for example, depending upon whether the particular user experiences frequent, or infrequent, upper gastrointestinal tract conditions).

In another embodiment, the kit is comprised of a recloseable container that contains two inner containers, each inner container in turn comprising a sufficient number of unit doses of the gastric acid secretion inhibitor to comply with a given dosing period and instructions for complying with the treatment method; in yet another example, each inner container contains 14 unit doses of the composition and the kit contains a total of 28 unit doses of the composition (14 unit doses in each of 2 inner containers). In another embodiment, the kit is comprised of a recloseable container that contains three inner containers, each inner container in turn comprising a sufficient number of unit doses to comply with one dosing period and instructions for complying with the treatment method; in yet another example, each inner container contains 14 unit doses of the composition and the kit contains a total of 42 unit doses of the composition (14 unit doses in each of 3 inner containers). In another embodiment, the kit is comprised of a recloseable container that contains four inner containers, each inner container in turn comprising a sufficient number of unit doses to comply with one dosing period and instructions for complying with the treatment method; in yet another example, each inner container contains 14 unit doses of the composition and the kit contains a total of 56 unit doses of the composition (14 unit doses in each of 4 inner containers).

In another embodiment, the unit doses are contained in a blister pack. A blister pack may comprise a substrate containing one or more blisters, wherein each blister may contain a unit dose of the gastric acid secretion inhibitor or other medicament. Alternatively or additionally, each inner container comprises and/or contains at least one blister pack, wherein each blister pack contains at least one unit dose. For example, fourteen unit doses may be contained in two blister packs, each blister pack containing seven unit doses. In one embodiment of this type, two, three, or four (for example) inner containers each comprise two of the blister packs, wherein each of the blister packs comprises seven unit doses. In another embodiment, fourteen unit doses are contained in one blister pack, each blister pack containing fourteen unit doses. In one embodiment of this type, two, three, or four (for example) inner containers each comprise one of the blister packs, wherein each of the blister packs comprises fourteen unit doses.

As used herein, the term “instructions” refers to printed material that sets forth a description of how the user is to comply with the methods of the present invention (e.g., the discontinuous schedule). Such instructions may include descriptions through words, pictures, symbols, and/or other visible descriptors. Such direction need not utilize the actual words used herein, for example, “treatment”, “gastrointestinal,” “gastrointestinal tract condition”, “dosing period,” “evaluation period,” or the like, but rather use of words, pictures, symbols, and the like reasonably conveying same or similar meanings are contemplated within the scope of this invention.

Variants in terms of composition used, length of various dosing periods, and the like are set forth above with respect to the method described herein below and are all incorporated into the descriptions of the kits.

### Methods Optionally Used in Accordance With the Present Invention

In certain aspects, embodiments shown and described herein may relate to a method of treating upper gastrointestinal conditions in mammals, preferably in humans, in need of such treatment. The method comprises administering a composition comprising the gastric acid secretion inhibitor in accordance with a discontinuous schedule, which includes a first dosing period, a first evaluation period, and a second dosing period. During each dosing period, a plurality of unit doses of a composition containing a therapeutically effective amount of a gastric acid secretion inhibitor is administered.

“Administering,” “administer,” or the like, as used herein, refers to any means of introducing a therapeutic amount of a gastric acid secretion inhibitor to the subject in need thereof. One example is an oral administration. As used herein, the term “administration,” “administering,” or the like with respect to the mammal means that the mammal is administered, is directed to administer or, with reference specifically to “oral administration,” or “orally administering,” ingests or is directed to ingest, one or more compositions described herein. Wherein the mammal is directed to ingest one or more of the compositions, such direction may be that which instructs and/or informs the user that use of the composition may and/or will provide one or more general health and/or general physiological benefits including, but not limited to, treatment of an upper gastrointestinal tract condition. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or containing devices associated with the composition (e.g., a label present on a package containing the composition). See e.g., the kits described herein.

As used herein, “treat,” “treating,” “treatment” or the like means that administration of the referenced composition prevents, alleviates, ameliorates, inhibits, or mitigates one or more symptoms of the condition or the condition itself, or any like benefit with respect to the gastrointestinal tract condition in a mammalian subject in need thereof, particularly in humans. As such, this includes, for example: preventing an upper gastrointestinal condition from occurring in a mammal, for example when the mammal is predisposed to acquiring the upper gastrointestinal condition, but has not yet been diagnosed with the disease; inhibiting the upper gastrointestinal condition; and/or alleviating, reversing, or curing the upper gastrointestinal condition. Insofar as the methods are directed to preventing an upper gastrointestinal condition, it is understood that the term “prevent” does not require that the upper gastrointestinal condition be completely thwarted. Rather, as used herein, the term “preventing” or the like refers to the ability of the skilled artisan to identify a population that is susceptible to upper gastrointestinal conditions, such that administration of the referenced compositions may occur prior to the onset of the symptoms of the condition.

As used herein, one discontinuous schedule comprises, in the following order, a first dosing period, a first evaluation period, and a second dosing period. In an optional embodiment, the discontinuous schedule may comprise a second evaluation period and further, optionally, a third dosing period. In yet another embodiment, the discontinuous schedule may comprise even further alternating evaluation and dosing periods.

As used herein, “dosing period” refers to a period of time within the discontinuous schedule during which a unit dose is administered, preferably once daily. The dosing period may comprise at least about 2 days, from about 2 to about 28 days, from about 5 to about 21 days, or from about 7 to about 14 days. The number of unit doses administered during each dosing period may be at least two, from 2 to about 28, from about 5 to about 21, or from about 7 to about 14. Optionally an identical number of unit doses is administered during each dosing period. Alternatively or additionally, it is optional that a unit dose is administered on consecutive days. However, it is neither required that an identical number of unit doses is administered during each dosing period (meaning, the dosing periods are independent of each other), nor that a unit dose be administered on consecutive days. The dosing periods begin with the administration of the first unit dose, and end upon administration of the last unit dose. Each dosing period, therefore, need not, but may be, identical in length.

As used herein, “evaluation period” refers to a period within a discontinuous schedule during which no unit doses are administered. An optional purpose of the evaluation period is to ensure that the gastric acid secretion inhibitor is effectively administered for over-the-counter use. Another purpose of the evaluation period is to give the user the opportunity to assess his or her symptoms. If symptoms do not recur, then the user may conclude that no additional dosing period is required. A given evaluation period comprises at least two days, more preferably from about 45 days to about 135 days, more preferably from about 75 days to about 135 days, and most preferably from about 90 days to about 125 days.

In one embodiment of this aspect, the discontinuous schedule comprises a first dosing period, a first evaluation period, a second dosing period, and a second evaluation period. In this embodiment, the first evaluation period is subsequent to the first dosing period and precedes the second dosing period, the second dosing period is subsequent to the first evaluation period and precedes the second evaluation period, and the second evaluation period is subsequent to the second dosing period and precedes any third dosing period or any other dosing or evaluation periods. Further dosing periods, alternating between evaluation periods are optional. Further evaluation periods, alternating between dosing periods are optional. The duration of all dosing and evaluation periods may be independent relative to the length of time of any other period.

As used herein, “subsequent to” refers to directly following the prior dosing period in time. As used herein, “preceding” refers to being directly prior to the time during which a new dosing period may begin. For example, administration of the final unit dose of a composition during a given dosing period marks the end of such dosing period and the beginning of the evaluation period.

Therefore, one embodiment of a discontinuous schedule provides for a first dosing period during which from about 10 to about 14 unit doses of a composition are administered, a first evaluation period from about 90 to about 125 days, and a second dosing period during which from about 10 to about 14 unit doses of a composition are administered.

Another embodiment herein provides for a first dosing period of about 10 to about 14 days, during which a total of about 10 to about 14 unit doses are administered individually once daily, followed by a first evaluation period from about 90 to about 125 days, followed by a second dosing period of about 10 to about 14 days, during which a total of about 10 to about 14 unit doses are administered individually once daily.

Another embodiment herein provides for a first dosing period of about 10 to about 14 days, during which a total of about 10 to about 14 unit doses are administered individually once daily, followed by a first evaluation period from about 90 to about 125 days, followed by a second dosing period of about 10 to about 14 days, during which a total of about 10 to about 14 unit doses are administered individually once daily, followed by a second evaluation period from about 90 to about 125 days, followed by a third dosing period of about 10 to about 14 days, during which a total of about 10 to about 14 unit doses are administered individually once daily.

### EXAMPLES

One example of a kit includes an outer container, such as, for example, a recloseable container as shown and described herein. The outer container contains two inner containers, each in the form of boxes that together fit inside the recloseable container. Each separate inner container in turn contains two blister packs, with 7 unit doses of an aforementioned composition per blister pack (for example, the composition may comprise about 20 mg of an omeprazole magnesium salt). Therefore, each separate inner container contains 14 unit doses of the composition, and the kit as a whole contains a total of 28 unit doses of the composition. Each separate inner container also contains a folded piece of paper with written instructions for compliance with the described treatment method (alternatively in this example, each separate inner container has these written instructions printed on a surface of the inner container). The instructions read, in part, as follows:

### Course of Treatment:

Take every day for 14 days.
Do not take more than 1 tablet a day.
You may repeat a 14-day course of therapy every 4 months.

A second example of a kit includes an outer container such as, for example, a recloseable container as shown and described herein. The outer container contains three separate inner containers, each in the form of blister packs that together fit inside the outer container. Each separate inner container in turn contains one blister pack, with 14 unit doses per blister pack. Therefore, each separate inner container contains 14 unit doses of an aforementioned composition per blister pack (for example, the composition may comprise about 20 mg of an omeprazole magnesium salt) and the kit contains a total of 42 unit doses of the composition. Each separate inner container also contains a folded piece of paper with written instructions for compliance with the described treatment method (alternatively in this example, each separate inner container has these written instructions printed on a surface of the inner container).

Any of the foregoing kits may include a recloseable container housing one or more inner containers. As used herein, “housing” and “housed” refers to an inner container being removably disposed within the confines of the recloseable container. For example, the recloseable container can comprise a reservoir disposed within the recloseable container that is at least partially defined by a front wall and a front flap, such that each of the one or more inner containers can be removed from the reservoir when the front flap is not in a substantially closed position with respect to the front wall. Referring to FIGS. 4-7, a single recloseable container 10 may house a one or more inner containers 800 wherein each of the inner containers 800 contains at least one unit dose. In one embodiment, for example, each of the inner containers 800 may comprise a blister pack containing seven or fourteen unit doses. Instructions may be placed on the inside of the recloseable container 10 or on each of the inner containers 800 as will be discussed herein. Without intending to be bound by a particular theory, the recloseable aspect of the recloseable container 10 may combine with the instructions to queue and/or assist the subject in alternating between dosing periods and evaluation periods as discussed herein.

Referring now specifically to FIG. 4, the recloseable container 10 is shown in a sealed position as will be described below herein. In one embodiment, the recloseable container 10 may generally comprise a front flap 500 that may be disposed outside of a front wall 100 of the recloseable container 10. One or more anchor portions 590 may be disposed about the front wall 100 and releasably connect to the front flap 500 when the front flap 500 is in the sealed position as depicted in FIG. 4. For example, in the embodiment illustrated in FIGS. 4-7, two anchor portions 590 may be connected to the bottom two comers of the front wall 100. The anchor portions 590 may be secured to the exterior of the front wall 100 via adhesives and may conform to the shape of the flap 500 to allow for a smooth fit between the front flap 500 and anchor portions 590. The front flap 500 may thereby be releasably connected to the anchor portions 590 along a connection line 580. For example, the connection line 580 may comprise a perforated connection between the flap 500 and anchor portions 590, may comprise a pull-tab type connection between the flap 500 and anchor portions 590, or may comprise any alternative structure allowing for the releasable connection between the flap 500 and the anchor portions 590 such that the flap 500 may transition from the sealed position (as shown in FIG. 4) to an open position (as shown in FIGS. 5 and 6). In an alternative embodiment, the flap 500 may form a releasable connection directly with the front wall 100 or other part of the recloseable container 10 without the aid of additional anchor portions 590.

The front flap 500 may further comprise a tab 550 that may interact with a slit 150 to permit the closing of the recloseable container 10 after the flap 500 has been unsealed from the anchor portions 590 (or front wall 100 in an alternative embodiment) and thus to assist in closing the recloseable container 10 when the front flap 500 transitions between the open position (as seen in FIGS. 5 and 6) and a closed position (as seen in FIG. 7). For example, referring to FIGS. 5-7 specifically, the tab 550 may comprise an appendage-like protrusion from the front flap 500 that is operable to fit into the slit 150 on the front wall 100. The slit 150 may comprise a gap in the front wall 100 operable to receive the tab 550 such that when the front flap 500 is in the closed position (as seen in FIG. 7), the tab 550 inserts into the slit 150 and rests on the inside of the recloseable container 10 holding the front flap 500 against the front wall 100. The slit 150 and tab 550 may comprise any shape or geometry such that the tab may be inserted and removed from the slit to effectively hold the front flap 500 against the front wall 100. For example, in one embodiment, as seen in FIGS. 4-7, the slit 150 may comprise a “smile-like” configuration with the inside of the concave geometry facing upwards towards the top of the recloseable container 10. The tab 550 may further comprise a downward facing protrusion with a narrowing tip 551 for insertion into the slit 150. In another embodiment, the front flap 500 may comprise a continuous edge with no definable protrusion such that a portion of the continuous edge itself enters the slit 150 when in the closed position.

In one embodiment, the slit may further comprise one or more lateral support cuts 155 adjacent the slit 150 to assist in the closure of the front flap 500. The lateral support cuts 155 may comprise a cut in the front wall 100 that extends outwards from the slit 150 such that the tab 550 and/or the front flap 500 may be tucked behind a bottom portion 157 of the slit 150 when the tab 550 is inserted into the slit 150 and thus placing the flap 500 in the closed position as seen in FIG. 6. In one embodiment, two lateral support cuts 155 may first extend from opposite sides of the slit 150 in a substantially downward direction before extending in an upward direction. Such an embodiment may provide additional clearance for the front flap 500 to tuck into the front wall 100 and behind the bottom portion 157 of the slit 150 when the tab 550 is inserted into the slit 150.

Referring now specifically to FIG. 6, the recloseable container 10 may store or hold one or more inner containers 800, each comprising a plurality of unit doses 850. Each inner container 800 may provide and represent the corresponding number of unit doses 850 required for a certain dosing period. For example, in one embodiment, each inner container 800 may comprise a blister pack containing 14 unit doses 850. As such, each inner container 800 (i.e., blister pack) provides for a single dosing period containing or consisting of 14 unit doses 850. In turn, the recloseable container 10 may hold three inner containers 800, thereby providing for three separate dosing periods within the recloseable container 10. In other embodiments, any other number of inner containers 800 containing any number of unit doses 850 may be contained in the recloseable container 10 as alternatively discussed above. A user may thereby remove an individual inner container 800 for a specific dosing period from the recloseable container 10 and subsequently close the recloseable container 10 to isolate the given dosing period's unit doses from the remaining unit doses stored in the recloseable container 10. Without intending to be bound by a particular theory, such a recloseable container housing separate inner containers for separate dosing periods provides a clear delineation of the starting and stopping point for each dosing period and thus provides some additional queues and signals to the subject that each inner container 800 comprises units doses for separate dosing periods, wherein an evaluation period is recommended and/or required in between each dosing period.

Instructions may be disposed on various locations of the recloseable container 10 and/or on the inner container(s) 800 stored therein to further remind the subject to engage in an evaluation period between dosing periods or otherwise assist in conforming with the treatment methods described herein. In one embodiment, instructions may be disposed about an interior surface of the recloseable container 10. Interior surface may refer to any surface that is only visible to the subject when the recloseable container is in a substantially open position. For example, still referring to FIG. 6, in one embodiment, instructions may be disposed about an inner surface 510 of the front flap 500 of the recloseable container 10 such that the subject may see and be reminded of the instructions every time an inner container 800 is removed from the recloseable container 10. In another embodiment, instructions may be disposed about the front wall 100 of the recloseable container 10, or in particularly about the portion of the front wall 100 revealed when the front flap 500 is opened such as surface area 110. In yet another embodiment, instructions may be disposed about each individual inner container 800 contained in the recloseable container 10. Instructions may be disposed in a single location or a plurality of locations so that the subject may see the instructions before removal of an inner container 800 from the recloseable container 10. In addition, the configuration of the inner container being recloseable in and of itself provides a queue and/or signal to the subject in that the act of opening the recloseable container to obtain a new inner container comprising unit doses for new dosing period provides a queue and/or signal, as well as another opportunity to view the instructions, that an evaluation period is required in between each dosing periods.

Referring now to FIG. 8, in one embodiment, the recloseable container may be constructed from a blank 1000. The blank 1000 may generally comprise a front wall 100 connected to a first side 201 of a back wall 200 by a bottom wall 310, and a front flap 500 connected to a second side 202 of the back wall 200, opposite the first side, by a top flap 320. The blank 10 may be transitioned into a constructed form of the recloseable container. For example, the front wall 100 and back wall 200 may be folded upwards about the bottom wall 200 such that the front wall 100 and back wall 200 become substantially parallel with one another. The front flap 500 may then be folded downwards about the top wall 320 such that the front flap 500 becomes substantially parallel with both the front wall 100 and back wall 200 and rests on the outside of the front wall 100. The front flap 500 may then be secured to the front wall 100 such that the recloseable container maintains a sealed configuration. For example, as discussed above, two anchor portions may be connected to the front flap 500 via perforations and may be secured to the front wall via adhesives, tabs, seals or the like. Thus, the flap may be pulled away from the anchor portions to transition the recloseable container from a sealed position to an open position.

A plurality of side walls may further be disposed about the blank 10 to enclose an interior of the recloseable container when constructed. In one embodiment, side walls may be disposed on two sides of the front wall 100, bottom wall 310, back wall 200 and top wall 320. For example first and second side flaps 331,332 may be connected to the front wall 100 on opposite sides of the front wall, and third and fourth side flaps 335,336 may be connected to the back wall 200 on opposite sides of the back wall. When the blank is constructed into a recloseable container, the first side flap 331 may combine with the third side flap 335 to close off one side of the recloseable container. Likewise, the second side flap 332 and fourth side flap 336 may similarly combine to close off the other side of the recloseable container. The side flaps may combine to become secured with one another through adhesives, bonding, tabs or any alternative mode of fixedly connecting the two flaps as known to one of ordinary skill in the art. In another embodiment, side walls may only be disposed on one side of the front wall 100, bottom wall 310, back wall 200 and top wall 320. For example, the side walls may wrap around and secure to the opposite wall such that only one side flap is needed to traverse and enclose the gap between the front wall 100 and back wall 200. The blank may further comprise additional dust flaps to assist in adequately sealing the interior of the recloseable container when in the sealed or closed position. For example, first and second dust flaps 333,334 may be disposed on opposite sides of the bottom wall 310 while third and fourth dust flaps 337,338 may be disposed on opposite sides of the top wall 320. The dust flaps may thereby be tucked into the interior of the recloseable container to provide for further separation of the interior from outside elements.

Instructions may be disposed about one or more locations of the blank 10 to further remind the subject to engage in an evaluation period between dosing periods or otherwise assist in conforming with the treatment methods described herein. For example, instructions may be disposed about the front wall 100 or an inside surface of front flap 500.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A kit comprising:
a. a recloseable container that comprises:
(a) a front wall; and
(b) a front flap operable to transition between an open position and a closed position, wherein the front flap releasably engages the front wall when in the closed position; and
b. an inner container housed within the recloseable container wherein the inner container comprises:
(a) a plurality of unit doses of a composition comprising a therapeutically effective amount of a gastric acid secretion inhibitor; and
(b) instructions for compliance with a course of treatment comprising:
1. that at least 2 unit doses of the composition should be administered over a dosing period from about 2 days to about 28 days; and
2. that the course of treatment may be repeated after an evaluation period from about 2 days to about 135 days.

2. The kit of claim 1 wherein the gastric acid secretion inhibitor is omeprazole.

3. The kit of claim 2 wherein the kit comprises a plurality of inner containers housed within the recloseable container.

4. The kit of claim 3 wherein the evaluation period comprises from about 75 days to about 135 days.

5. The kit of claim 4 wherein the evaluation period is about 4 months.

6. The kit of claim 5 wherein the unit doses are administered once daily during each of the dosing periods.

7. The kit of claim 6 wherein the therapeutically effective amount is from about 5 mg to about 80 mg.

8. The kit of claim 7 wherein the unit doses are in a form selected from the group consisting of tablets and capsules.

9. The kit of claim 8 wherein the omeprazole is an omeprazole salt.

10. The kit of claim 8 wherein each inner container comprises a blister pack comprising 14 blisters, each blister containing a unit dose of the composition.

11. A kit comprising:
a. a recloseable container that comprises:
(a) a front wall;
(b) a front flap operable to transition between an open position and a closed position, wherein the front flap releasably engages the front wall when in the closed position and wherein an interior surface of the recloseable container comprises instructions for compliance with a course of treatment comprising:
1. that at least 2 unit doses of a composition comprising a therapeutically effective amount of a gastric acid secretion inhibitor should be administered over a dosing period of from about 2 days to about 28 days, and
2. that the course of treatment may be repeated after an evaluation period of from about 2 days to about 135 days;
(c) a reservoir disposed within the recloseable container and at least partially defined by the front wall and front flap; and
(d) an inner container housed within the reservoir wherein the inner container comprises a plurality of unit doses.

12. The kit of claim 11 wherein the plurality of unit doses of the inner container contain a gastric acid secretion inhibitor which is omeprazole.

13. The kit of claim 12 wherein the kit comprises a plurality of inner containers housed within the recloseable container and wherein each of the plurality of inner containers comprises a plurality of unit doses containing a therapeutically effective amount of the omeprazole.

14. The kit of claim 13 wherein the evaluation period comprises from about 75 days to about 135 days.

15. The kit of claim of 14 wherein the evaluation period is about 4 months.

16. The kit of claim 15 wherein the unit doses are administered once daily during each of the dosing periods.

17. The kit of claim 16 wherein the interior surface comprising the instructions is an inside surface of the front flap.

18. The kit of claim 16 wherein the interior surface comprising the instructions is a surface of the front wall that is covered by the flap when in the closed position.

19. The kit of claim 16 wherein the interior surface comprising the instructions is a surface on the inner container.

20. The kit of claim of 16 wherein the therapeutically effective amount is from about 5 mg to about 80 mg.

21. The kit of claim of 20 wherein the unit doses are in a form selected from the group consisting of tablets and capsules.

22. The kit of claim of 21 wherein the omeprazole is an omeprazole salt.

23. The kit of claim of 21 wherein the inner container comprises a blister pack comprising 14 blisters, each blister containing a unit dose of the composition.

24. A method for administering a plurality of dosing periods with evaluation periods there between, the method comprising:
a. providing a recloseable container housing an inner container, wherein the inner container comprises a plurality of unit doses of a composition comprising a therapeutically effective amount of a gastric acid secretion inhibitor; and
b. providing on an interior surface of the recloseable container instructions for compliance with a course of treatment comprising:
(a) that at least 2 unit doses of the composition should be administered over a dosing period of from about 2 days to about 28 days, and
(b) that the course of treatment may be repeated after an evaluation period of from about 2 days to about 135 days.

25. The method of claim 24 wherein the gastric acid secretion inhibitor is omeprazole.

26. The method of claim 25 wherein the interior surface comprises an inside surface of a front flap of the recloseable container.

27. The method of claim 25 wherein the interior surface comprises a portion of a front wall of the recloseable container that is covered by a recloseable flap of the recloseable container when the recloseable flap is in a closed position.

28. The method of claim 25 wherein the interior surface comprises a portion of a surface of the inner container.

29. The method of claim 25 wherein the interior surface comprise a portion of the recloseable container and a portion of the inner container such that the instructions for compliance with a course of treatment are disposed on the portion of the recloseable container and the portion of the inner container.

30. The method of claim 25 wherein the kit comprises a plurality of inner containers housed within the recloseable container.

31. The method of claim 25 wherein the evaluation period comprises from about 75 days to about 135 days.

32. The method of claim of 31 wherein the evaluation period is about 4 months.

33. The method of claim 32 wherein the unit doses are administered once daily during each of the dosing periods.

34. The method of claim of 33 wherein the therapeutically effective amount is from about 5 mg to about 80 mg.

35. The method of claim of 34 wherein the compositions are in a form selected from the group consisting of tablets and capsules.

36. The method of claim 35 wherein the omeprazole is an omeprazole salt.

37. The method of claim of 36 wherein each package of the plurality of packages comprises a blister pack comprising 14 blisters, each blister containing a unit dose of the composition.

38. A blank for a recloseable container, the blank comprising:
a. a front wall connected to a first side of a back wall by a bottom wall;
b. a front flap connected to a second side of the back wall opposite the first side by a top wall, the front flap operable to removably engage a slit in the front wall when in a constructed position; and
c. instructions for compliance with a course of treatment comprising:
(a) that at least 2 unit doses of a composition comprising a gastric acid secretion inhibitor should be administered over a dosing period of from about 2 days to about 28 days; and
(b) that the course of treatment may be repeated after an evaluation period of from about 2 days to about 135 days.

39. The blank of claim 38 wherein the instructions are disposed about an inside surface of the front flap.

40. The blank of claim 38 wherein the instructions are disposed about the front wall.

41. The blank of claim 38 wherein the slit comprises one or more lateral support cuts.
